(19) United States
(12) Patent Application Publication
(10) Pub. No.: US 2022/0028544 A1
(43) Pub. Date: Jan. 27, 2022

(54) SYSTEM AND METHOD FOR PROVIDING URGENT CARE

(71) Applicant: DocV Inc., Boston, MA (US)

(72) Inventors: David R. ELMALEH, Newton, MA (US); Pinhas COHEN, Copper City, FL (US)

(73) Assignee: DocV Inc., Boston, MA (US)

(21) Appl. No.: 17/384,144

(22) Filed: Jul. 23, 2021

Related U.S. Application Data
(60) Provisional application No. 63/056,485, filed on Jul. 24, 2020.

(51) Int. Cl. G16H 40/67; G16H 80/00; H04W 4/02; G06Q 10/10; G06Q 20/10

(52) U.S. Cl. CPC G16H 40/67; G16H 80/00; G06Q 20/102; H04W 4/025; G06Q 10/1095; H04W 4/023

(57) ABSTRACT

An urgent care provider system may comprise a server that is configured to: receive a request for medical assistance for a patient from a user mobile device that is registered with the system; identify one or a plurality of healthcare professionals that are registered with the system and who can provide the requested medical assistance; determine a location of the patient; determine locations of one or a plurality of healthcare professional mobile devices, each healthcare professional mobile device associated with a healthcare professional of the one or a plurality of healthcare professionals; calculate a travel time between each of the one or a plurality of healthcare professionals and the patient; present one or a plurality of possibilities of provision of the medical assistance by one or more of the one or a plurality of healthcare professionals; and, if one of the plurality of possibilities is selected, send to the healthcare professional mobile device that is associated with the selected healthcare professional instructions to provide the requested medical assistance to the patient.

### CROSS REFERENCE TO RELATED APPLICATION

This application claims priority from U.S. Provisional Patent Application No. 63/056,485, filed Jul. 24, 2020, which is incorporated herein by reference in its entirety.

### FIELD OF THE INVENTION

The present invention relates to a system for the provision of medical care. More particularly, the present invention relates to a system for providing urgent care to a patient.

### BACKGROUND OF THE INVENTION

Typically, different types of medical resources are available for different types of health issues. For example, in the case of routine health care, and for minor and chronic health issues in which treatment by a medical professional is not urgent (e.g., where a delay of a day or two is unlikely to adversely affect the patient's condition), a visit to a health clinic or physician's office may be sufficient. At the other extreme, in the case of emergency health care and life-threatening emergencies, where even a short delay may be serious, the patient may be brought to a hospital emergency room or an ambulance or emergency medical service may be called. Urgent health care may be used for issues that are not immediately life threatening but for which, e.g., for the patient's comfort or to avoid adverse effects, avoidance of emergency medical service is desired may be provided by bringing the patient to an urgent care center or to a medical clinic. Such emergency use of these facilities may contribute to increasing the cost and congestion of these facilities.

### SUMMARY OF THE INVENTION

In accordance with certain embodiments of the invention, an urgent care provider system comprises a server that is configured to: receive a request for medical assistance for a patient from a user mobile device that is registered with the system; identify one or a plurality of healthcare professionals that are registered with the system and who can provide the requested medical assistance; determine a location of the patient; determine locations of one or a plurality of healthcare professional mobile devices, each healthcare professional mobile device associated with a healthcare professional of the one or a plurality of healthcare professionals; calculate a travel time between each of the one or a plurality of healthcare professionals and the patient; present one or a plurality of possibilities of provision of the medical assistance by one or more of the one or a plurality of healthcare professionals; and, if one of the plurality of possibilities is selected, send to the healthcare professional mobile device that is associated with the selected healthcare professional instructions to provide the requested medical assistance to the patient.

In some embodiments, the server is further configured to determine whether to send a healthcare professional of said one or a plurality of healthcare professionals to the location of the patient, or to send the patient to a facility associated with one of the one or a plurality of healthcare professionals. In some embodiments, the server is configured to determine a time of an available appointment at the facility.

In some embodiments, the server is configured to determine the location of the patient by determining a location of the user mobile device.

In some embodiments, the server is configured to determine the location of the patient by receiving a location that is indicated by operation of the user mobile device.

In some embodiments, the server is configured to present the one or a plurality of possibilities by presenting the request for assistance concurrently to a plurality of the healthcare professional mobile devices.

In some embodiments, the server is configured to present the one or a plurality of possibilities by presenting the request for assistance sequentially to each of a plurality of the healthcare professional mobile devices until the request is via a healthcare professional mobile device of the plurality of the healthcare professional mobile devices.

In some embodiments, the server is configured to present the one or a plurality of possibilities by presenting offers of assistance one or a plurality of the healthcare professionals to the user mobile device.

In some embodiments, the server is further configured to request from the user mobile device data that is related to a medical condition of the patient. In some embodiments, the data comprises a photograph or a video recording of the patient. In some embodiments, the data comprises a temperature of the patient.

In some embodiments, the server is further configured to enable payment of a fee for provided medical assistance via the user mobile device.

### BRIEF DESCRIPTION OF THE DRAWINGS

In order for the present invention to be better understood and for its practical applications to be appreciated, the following Figures are provided and referenced hereafter. It should be noted that the Figures are given as examples only and in no way limit the scope of the invention. Like components are denoted by like reference numerals.

FIG. 1 is block diagram schematically illustrating an urgent care provider system, in accordance with an embodiment of the invention;

FIG. 2 is a flowchart depicting an example of a method of operation of a server of an urgent care provider system, in accordance with an embodiment of the invention; and

FIG. 3 is a flowchart depicting an example of a method of operation of a server of an urgent care system that includes directing a patient to the location of a healthcare professional.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, the conjunction “or” as used herein is to be understood as inclusive (any or all of the stated options).

Some embodiments of the invention may include an article such as a computer or processor readable medium, or a computer or processor non-transitory storage medium, such as for example a memory, a disk drive, or a USB flash memory, encoding, including or storing instructions, e.g., computer-executable instructions, which when executed by a processor or controller, carry out methods disclosed herein.

In accordance with an embodiment of the invention, an urgent care provider system is configured to locate one or more healthcare professionals (e.g., physicians, nurses, emergency medical technicians, paramedics, or other healthcare professionals) that are available to provide urgent health care in a timely manner to a patient. A mobile phone, or other portable device with a capability of determining a current location of the device, is associated with the patient. Any such mobile phone or portable device is referred to herein as a mobile device.

For example, the mobile device may belong to the patient or to another user (e.g., relative, coworker, caregiver, acquaintance, passerby, or other person) that is currently assisting the patient. The mobile device may be programmed with, or otherwise configured to run, an application or program that is associated with an urgent care provider service. Similarly, each healthcare professional that is registered with the service may be associated with a mobile device that is configured to run a similar application or program that is associated with the service.

When the patient or user that is requesting medical assistance activates the application, the user may be asked to either identify the type of healthcare professional required or the type of medical assistance required. In some cases, the user may indicate a medical condition for which care is required. In this case, the system may utilize a database, algorithm or other method to identify one or more types of healthcare professional that are appropriate for treatment of the indicated medical condition. The system may then identify the location of the mobile device from which the request for medical assistance was placed. In some cases, the system may determine that the indicated medical condition does not require urgent care, or that it requires immediate emergency care, and may direct the user to an alternative care provider. In some cases, the user may be enabled to specify a location of the patient that is different from that of user's mobile device (e.g., where service is being requested for a patient who is not currently with the user).

The system may then identify appropriate healthcare professionals that are registered with the service and the location of a mobile device that is associated with each. The identification of healthcare professionals may be limited to those healthcare professionals who have indicated their availability to provide services at the time of the request and at locations that include the location of the user. For example, each registered healthcare professional may indicate standing preferences, or variable preferences, regarding hours during which he/she is available to travel to a patient's location.

The system may present the request and location of the patient to one or more of the healthcare professionals, either concurrently or sequentially (e.g., in order of nearness to the patient, in the order in which the mobile devices of the healthcare professionals are contacted, or in another order).

Alternatively or in addition (e.g., concurrently to presenting to the healthcare professionals), the system may then report to the user in one or more forms (e.g., a list, markings on a map, or otherwise) identifying information regarding each of the identified healthcare professionals and the location of each. For example, each healthcare professional may be identified by name and by a title or other brief description of the healthcare professional's profession and specialty.

In some cases, the user may be enabled to access more detailed information regarding each identified healthcare professional (e.g., associations with institutions, period of time practicing a specialty, or other relevant information). In some cases, a photograph of each healthcare professional may also be provided (e.g., in order to assist the user in identifying the healthcare professional upon arrival). In some cases, the user may access reviews by previous patients of each healthcare professional.

The system provides a distance of each identified healthcare professional from the user's mobile device. For example, the mobile device of each healthcare professional that is registered with the urgent care provider service may be programmed to transmit its location to the system at fixed intervals (e.g., every five seconds, or at other intervals). The location of the mobile device may be determined via the Global Positioning System (GPS) or another navigation system or method. The system may store and periodically update a database that includes the current position of the mobile device of each healthcare professional that is associated with the urgent care provider service.

The position may be represented on a three-dimensional sphere so as to avoid problems associated with projections of the three-dimensional surface of the earth onto a two-dimensional surface. For example, the surface of the earth may be divided into cells bounded by spherical geodesic edges, e.g., as enabled by the GoogleTM S2Geometry library. Each cell may be characterized by a unique identifier, which may be used as a key (e.g., a shard key) in a database. For example, the system may identify all healthcare professionals whose mobile devices are in cells within a given radius of the user's mobile device.

A routing module of the system may calculate an estimated travel time for each identified healthcare professional and the user's mobile device.

One or more requests for assistance may be presented to one or more healthcare professionals. The presentation may be either concurrent (e.g., in a list) or sequential. Alternatively or in addition, available healthcare professionals may be presented to the user, either concurrently or sequentially. Each presented request or healthcare professional may be accompanied by an indication of a distance between the healthcare professional and the patient. For example, the distance may be indicated by one or more of an estimated travel time from the healthcare professional's location to the location of the patient, by an estimate travel distance (e.g., along a network of roads), by an indicated position on a map, or otherwise. The system may also present the user with a price for provision of the requested medical service by each identified healthcare professional.

A healthcare professional may indicate willingness to respond to the request, e.g., subject to the user's acceptance. Alternatively, or in addition, the user may select one of the identified healthcare professionals to provide the required service. The system may then relay the patient information to the selected healthcare professional, as well as instructions for traveling to the location of the user's device. The healthcare professional may then acknowledge the request for service and indicate intention to provide the service.

In some examples, the system may send a request to a single healthcare professional, e.g., that is closest to, or with the shortest calculated travel time from, the location of the user's device or to an otherwise selected healthcare professional. The healthcare professional may be required to indicate a willingness to accept the request within a limited period of time (e.g., one minute, two minutes or another period of time). If no response is received from the healthcare professional during that limited time period, the request may be sent to the next nearest healthcare professional, while optionally canceling the request that was sent to the first selected healthcare professional.

In some cases, the selected healthcare professional may indicate unavailability or unwillingness to provide the service immediately (e.g., due to another urgent matter), and may indicate when that healthcare professional expects to be available to provide the requested service. In this case, the user may be informed of the delay and may be enabled to either accept the delay or select a different healthcare professional.

The user may write a review of the selected healthcare professional, e.g., after the visit by the user to the healthcare professional. The review may be stored by the system and made available to future users of the system.

The application may also enable other services, such as making an appointment regarding a non-urgent matter, e.g., for an office visit, remote consultation, or other medical service.

For example, the application may locate one or more specialists or other requested healthcare professionals, e.g., at their offices or clinics, and may make, enable making or facilitate making an appointment with a selected healthcare professional. The application may determine the soonest appointment that is available with each healthcare professional, and the location of each healthcare professional. The application may also provide a price for a requested type of healthcare service as provided by each healthcare professional. The user may then select an appointment from the list of available appointments. Upon the user's selection of an appointment, the application will reserve the selected appointment for the user (or for a patient on whose behalf the user is requesting the healthcare service).

The purpose of the system as described herein is to improve the time to deliver urgent care to a patient in need. The system may, in some cases, assist in lowering the cost of healthcare to the patient. The system may improve the efficiency of providing urgent care. In some cases, the system may enable or initiate a telemedicine connection between a healthcare professional and a patient, e.g., to determine urgency for the purpose of triage. The system may be configured to send the healthcare professional to the patient or to send the patient to the healthcare professional, e.g., as needed or as requested by the patient or by the healthcare professional. In some cases, the system may be configured to automatically charge the user for any care that is provided.

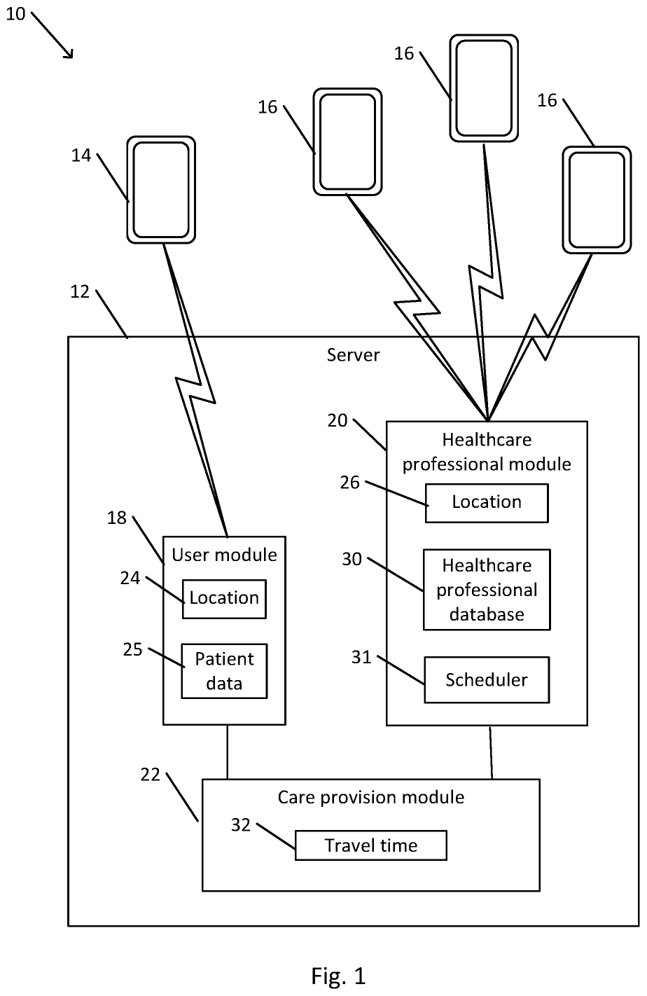

FIG. 1 is block diagram of an urgent care provider system, in accordance with an embodiment of the invention.

Urgent care provider system 10 includes a system server 12 that is configured to enable one or more users, each user associated with a user mobile device 14, to order services from a plurality of healthcare professionals, each healthcare professional being associated with a healthcare professional mobile device 16. For example, a user mobile device 14 or a healthcare professional mobile device 16 may include a cellular telephone that is capable of running an appropriate application (e.g., a user application or a healthcare professional application, respectively) that is associated with, and may be provided by, urgent care provider system 10. Each user mobile device 14 or healthcare professional mobile device 16 is capable of communication with other mobile devices and with system server 12. Each user mobile device 14 or healthcare professional mobile device 16 includes input and output capability to enable an operator of that device to enter and send data to system server 12 or elsewhere, and to communicate to the operator data or information received from system server 12 or elsewhere. Typically, input and output capability includes a touchscreen and audio communication (e.g., microphone and speaker).

The user may include a patient who requires the urgent care or a person who acts on behalf of the patient. A healthcare professional may include a physician, nurse, paramedic, medical technician, or other qualified provider of urgent healthcare.

System server 12 includes one or more processing units, e.g., in the form of computers or processing devices configured to operate in accordance with programmed instructions. The processing units may be located together, e.g., in a single housing or connected by cables, or may be remote from one another, e.g., intercommunicating via a wireless connection or a network (e.g., internet, mobile phone network, or other network).

The processing units of system server 12 are in communication with one or more data storage units, e.g., including fixed or removable, local or remote, volatile or nonvolatile data storage or memory devices. The data storage units may be utilized to store programmed instructions for operation of system server 12, data required for operation of system server 12, and results of operation of system server 12.

In the example shown, system server 12 is schematically represented as including three divided modules: user module 18, healthcare professional module 20, and care provision module 22. For example, each of user module 18, healthcare professional module 20, and care provision module 22 may represent a software module or a subset of functionality of system server 12. In some cases, each of user module 18, healthcare professional module 20 and care provision module 22 may represent different processing units or cooperating groups of processing units. Functionality of system server 12 may be represented as alternative division into modules or function.

User module 18 enables communication between user mobile device 14 and system server 12. For example, user module 18 may enable a user operating a user mobile device 14 to register with an urgent care provider service with which system server 12 is associated. When registering, the user may be provided with login information (e.g., a user name, a password, or other login information) in order to enable initiation of communication with system server 12. The user may be requested to provide billing information (e.g., credit card number, billing address, or other information), basic medical information (e.g., preexisting conditions, allergies, age, gender, or other information), information to expedite finding the user (e.g., addresses at which the user is frequently found, vehicle license, picture or identifying body features, or other information), or other information. The stored information may be saved in a user database.

When an operator of user mobile device 14 requests urgent care, either as a patient or on behalf of a patient (typically, although not necessarily, a relative, close acquaintance or personal representative of the user), system server 12 may request login information. The operator of user mobile device 14 may then enter a request for urgent care. For example, the operator may enter the type of provider required (e.g., medical specialty and whether a physician, nurse, or other type of provider is required), a description of the condition for which care is required (e.g., selected from a menu or list of conditions, or entered as a text or voice message), and, in the event that the patient is not the operator of user mobile device 14, identification of the patient (e.g., an identification number or code when the patient is registered with urgent care provider system 10, or other patient information by providing basic information in response to requests by system server 12). In the event that the patient is not then physically with the operator of user mobile device 14, the location of the patient may also be provided.

User module 18 includes a user location module 24. User location module 24 may enable system server 12 to communicate with user mobile device 14 to determine a location of user mobile device 14. User mobile device 14 typically includes a PS receiver or one or more other navigation devices or systems to determine a location of user mobile device 14 relative to one or more global or local coordinate systems (e.g., latitude and longitude, municipal street grid, or other coordinate system). User location module 24 may communicate with user mobile device 14 to receive the determined location of user mobile device 14. Alternatively, or in addition, user location module 24 may be configured to convert location data that is received from user mobile device 14 to a location in coordinates convenient to system server 12.

User module 18 includes patient data module 25. Patient data module may enable system server 12 to communicate with user mobile device 14 to determine the type and the urgency of care that is to be provided to the patient. For example, system server 12 may request answers to a series of one or more questions that enable determination of a preliminary diagnosis for the purpose of triage or in order to determine type of care required (e.g., whether to send the patient to the location of a healthcare provider or to send a healthcare provider to the location of the patient). Questions may include a description of pain, a location of pain, injury, bleeding, discoloration, or other visible features, a measured temperature of the patient, and data from any type of measuring devices (e.g., including but not limited to blood pressure and blood sugar level) that are in the possession of the user or patient. Patient data module 25 may enable the user to send data that are acquired by user mobile device 14, for example, photographs or video of an affected area of the patient's body, recording of the patient's speech, breathing, or heartbeat, or other types of recordable data.

When the user is the patient, basic patient data and medical history (e.g., age and other relevant personal data) are typically stored in a database of urgent care provider system 10. Similarly, in the case where the patient is already registered with urgent care provider system 10, a user need only send identifying information of the patient to urgent care provider system 10. In the case of a patient that is not registered with urgent care provider system 10, the user may be requested to enter more detailed information, and the user may be charged for any service that is rendered by urgent care provider system 10.

The request for urgent care, together with relevant details of the patient's condition, and the location data may be transmitted to care provision module 22.

Healthcare professional mobile device 16 of system server 12 is configured to communicate with a plurality of healthcare professional mobile devices 16 that are each associated with a healthcare professional who is registered with the urgent care provider service.

For example, healthcare professional mobile device 16 may enable a healthcare professional operating a healthcare professional mobile device 16 to register with an urgent care provider service with which system server 12 is associated. When registering, the healthcare professional may be provided with login information (e.g., a user name, a password, or other login information) to enable initiation of communication with system server 12. The healthcare professional may be requested to provide information regarding qualifications (e.g., specialty, associations with healthcare institutions and organizations, experience, education and licensing information), billing rates and information (e.g., account to which money is to be paid, tax-related information), home and work addresses, vehicles operated by the healthcare professional, or other information. An identifier of healthcare professional mobile device 16 may be obtained.

In some cases, the location may be presented in the form of an identifier of a geographic cell (e.g., geographic region bounded by spherical geodesic edges) that is characterized by a unique identifier.

The stored information regarding each healthcare professional may be saved in healthcare professional database 30. For example, healthcare professional database 30 may be organized according to one or more database architectures that may be utilized to facilitate searching for a suitable healthcare professional.

For example, requirements for the database may include horizontal scalability by linear addition of capacity by addition of database servers, write availability by use of buffering, ensuring that triggers continue to operate after a change in instance, and ensuring that all operations (e.g., expanding storage, backup, adding indices, adding data, and other operations) are always possible (no downtime). The architecture may include a load balancer to distribute tasks among available resources. Cloud computing services may be utilized to provide for backup and recovery from failures.

For example, care provision module 22 may determine which registered healthcare professionals match the requirements of a request for urgent care from a user mobile device 14. Healthcare professional location module 26 of healthcare professional module 20 may then enable system server 12 to communicate with healthcare professional mobile device 16 for each suitable healthcare professional to determine a location of each healthcare professional mobile device 16. The locations of each healthcare professional mobile device 16 that is associated with a healthcare professional who is a potential responder to the request for urgent care may be provided to care provision module 22.

Travel time module 32 of care provision module 22 may calculate a travel time between a location of each of one or more healthcare professional mobile devices 16 and the location of user mobile device 14 (or of a patient). For example, travel time module 32 may access one or more of a roadmap (e.g., a digitized map), traffic data, or other data to calculate the travel time.

In some cases, care provision module 22 may be configured to determine whether care is best provided by sending a healthcare professional to the location of the patient, or whether it is best provided by sending the patient to the location (e.g., office, clinic, hospital emergency room or department, or other location) of the healthcare professional (e.g., due to lack of urgency, need for specialized or immobile equipment, more efficient use of resources, or for other reasons). The determination may be made automatically, e.g., in accordance with predetermined criteria, or may be made by a trained human supervisor of urgent care provider system 10.

When care provision module 22 determines that the patient is to be sent to the location of a healthcare professional, care provision module 22 may send to user mobile device 14 a list of healthcare professionals and their locations. In addition, care provision module 22 may send to user mobile device 14 one or more available appointments at each healthcare professional location.

For example, healthcare professional module 20 may include a scheduler module 31. Scheduler module 31 may communicate with an appointment calendar that is associated with each healthcare professional mobile device 16 to find a nearest appointment.

In some cases, care provision module 22 may limit the available appointments that are sent to user mobile device 14 to those appointments to which the patient or user may travel to before the time of the appointment (e.g., with a predetermined additional time period), e.g., as determined by travel time module 32. In some cases, e.g., where the user indicates that the patient will travel using public transportation, travel time module 32 may consult one or more public transportation schedules in order to estimate the travel time. When acceptance of one of the appointments is received from the user, care provision module 22 may generate a calendar entry or other reminder, as well as details of the appointment and travel instructions, to be sent to user mobile device 14.

System server 12 may execute an urgent care provider method in order to find a suitable healthcare professional to provide urgent care to a patient.

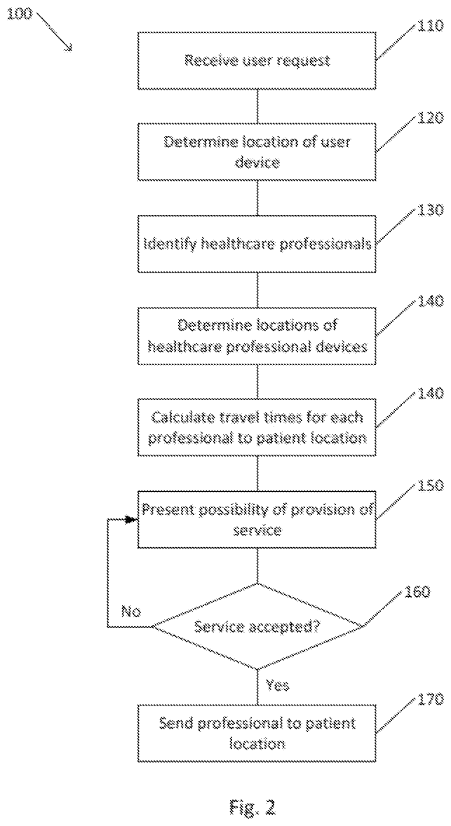

FIG. 2 is a flowchart depicting an example of a method of operation of a server of an urgent care provider system, in accordance with an embodiment of the invention.

It should be understood with respect to any flowchart referenced herein that the division of the illustrated method into discrete operations represented by blocks of the flowchart has been selected for convenience and clarity only. Alternative division of the illustrated method into discrete operations is possible with equivalent results. Such alternative division of the illustrated method into discrete operations should be understood as representing other embodiments of the illustrated method.

Similarly, it should be understood that, unless indicated otherwise, the illustrated order of execution of the operations represented by blocks of any flowchart referenced herein has been selected for convenience and clarity only. Operations of the illustrated method may be executed in an alternative order, or concurrently, with equivalent results. Such reordering of operations of the illustrated method should be understood as representing other embodiments of the illustrated method.

In particular, it should be noted that, in different embodiments or examples, operations that are indicated by some of the blocks of the flowchart may be skipped or may be made optional.

Urgent care provider method 100 may be executed by a processor of system server 12.

A request for medical assistance from a user mobile device 14 may be received (block 110). For example, the request may include a type of healthcare professional required, or a condition of a patient for which care is requested.

In some cases, system server 12 may send to user mobile device 14 a request for one or more types of login information. In this case, receipt of the request from user mobile device 14, as well as other operations of urgent care provider method 100, may be blocked or suspended until the login information is received. In some cases, system server 12 may send to user mobile device 14 a request for further information specifying the patient or the patient's condition, which may be received from user mobile device 14. For example, the request for further information may include a request for a more detailed description of the patient's condition, a photograph or video of a part of the patient's body that is related to the patient's condition, a recording of the patient, or other data.

System server 12 may communicate with user mobile device 14 to determine a location of user mobile device 14 (block 120). For example, system server 12 may communicate with user mobile device 14 to receive a latitude and longitude of user mobile device 14 (e.g., as determined by a GPS receiver of user mobile device 14), may receive GPS (or other navigation signals) that are received by user mobile device 14, or otherwise receive a location of user mobile device 14. In some cases, system server 12 may convert the location to an identifier of a geographic cell in a global arrangement of geographic cells that are each bounded by spherical geodesic edges. A geographic location of user mobile device 14 may be otherwise specified.

In some cases, the user of user mobile device 14 may specify, and system server 12 may receive, a location of a patient that is different from that of user mobile device 14. In this case, further reference to the location of user mobile device 14 in steps of urgent care provider method 100 may refer to the received location of the patient.

In some cases, the patient and details of the request may be added to a list of service requests.

System server 12 may identify one or more healthcare professionals that are available to provide the requested care and that are registered with the urgent care provider service that operates system server 12 (block 130). For example, system server 12 may select one or more healthcare professionals from healthcare professional database 30 whose profession or specialty corresponds to a profession or specialty that is requested by the user or that is matched with the user (e.g., via a database or algorithm that matches a described condition of the patient to a type of healthcare professional). Selection of healthcare professionals may be limited to those who are not currently providing care to a different user or patient. Selection of a healthcare professional may be further limited in accordance with limitations that are requested by each healthcare professional. For example, service that is provided by a particular healthcare professional may be limited to specific times (e.g., when that healthcare professional is not involved with other duties), to specific geographical boundaries, or other limitations.

The location of a healthcare professional mobile device 16 that is associated with each identified urgent care provider service may be determined (block 140).

The travel time from the location of each healthcare professional mobile device 16 to the location of the patient may be calculated (block 140). The calculation may be based on a distance by road, e.g., as calculated based on a roadmap that is available to system server 12, or to a route calculation service that is accessed by system server 12. The calculation may include typical traffic conditions, e.g., based on date and time of day, or may be based on current traffic conditions as obtained from a service that monitors traffic conditions.

One or more possible connections between an available healthcare professional and a patient may be presented (block 150). The connection may be accepted or rejected (block 160). If not accepted, one or more other possible connections may be presented (returning to block 150).

In some examples, requests for service from one or more user mobile devices 14 may be sent to one or more healthcare professional mobile devices 16.

In one example, a list of requests may be sent as a list to all healthcare professional mobile devices 16 that are associated with available healthcare professionals. Each healthcare professional may review the requests, including the estimated travel time and the nature of the request. When one of the healthcare professionals accepts the request, the offer of service may be sent to the requesting user mobile device 14, including a fee for the service. If acceptance of the offer is received by system server 12, the request for service may be removed from the list that is presented to all other healthcare professional mobile devices 16.

In another example, a single request or a list of the requests may be presented to one healthcare professional mobile device 16 at a time. If no response from that healthcare professional mobile device 16 is received within a predetermined period of time (e.g., one minute or another period, which may be determined by other conditions such as time of day or a current load of requests), the request or requests may be presented to another healthcare professional mobile device 16. This procedure may continue until a request is accepted by one of the healthcare professionals.

In some cases, a reason for not accepting a request may be noted (e.g., not requiring urgent care, requiring emergency care, or other reasons). In some cases (e.g., where it is important that the user or patient be made aware of the reason), the reason may be sent to user mobile device 14.

Alternatively, or in addition, a list of available healthcare professionals, including the estimated travel time to, and fee for, each may be presented to the requesting user mobile device 14 prior to presenting the request to healthcare professional mobile device 16. The user of user mobile device 14 may select a healthcare professional from the list. The request may then be sent to the healthcare professional mobile device 16 of the selected healthcare professional, who may accept or reject the request. If the request is rejected, the user of user mobile device 14 may select a different healthcare professional from the list.

When a connection has been made between a request for service and an available healthcare professional, the healthcare professional is then sent to the location of the patient (block 170). A message to healthcare professional mobile device 16 that sends the healthcare professional to the patient may include travel information (e.g., a suggested route).

When a healthcare professional is sent to a patient, that healthcare professional may be temporarily removed from the list of available healthcare professionals. After rendering the service to the patient, system server 12 may receive a message from healthcare professional mobile device 16 indicating completion of the urgent care. At this point, the healthcare professional may be returned to the list of available healthcare professionals. In addition, system server 12 may bill the user of user mobile device 14 and may credit an account of the healthcare professional.

In the event that the healthcare professional determines that treatment is required at the location of the patient, the patient or user may be required to indicate consent to that treatment, e.g., via user mobile device 14, prior to commencement of the treatment.

Once the urgent care has been provided, urgent care provider system 10 may prepare a report of the diagnosis and provided treatment. The report may also include details of the user's request, records of any indicated consent, follow-up instructions, or other information. The report may be provided to the patient or user, e.g., via user mobile device 14 or otherwise, may be sent a physician that is listed in a database of urgent care provider system 10 as associated with (e.g., regular physician to) the user or patient, or to another type of medical record (e.g., as listed in a patient profile of urgent care provider system 10).

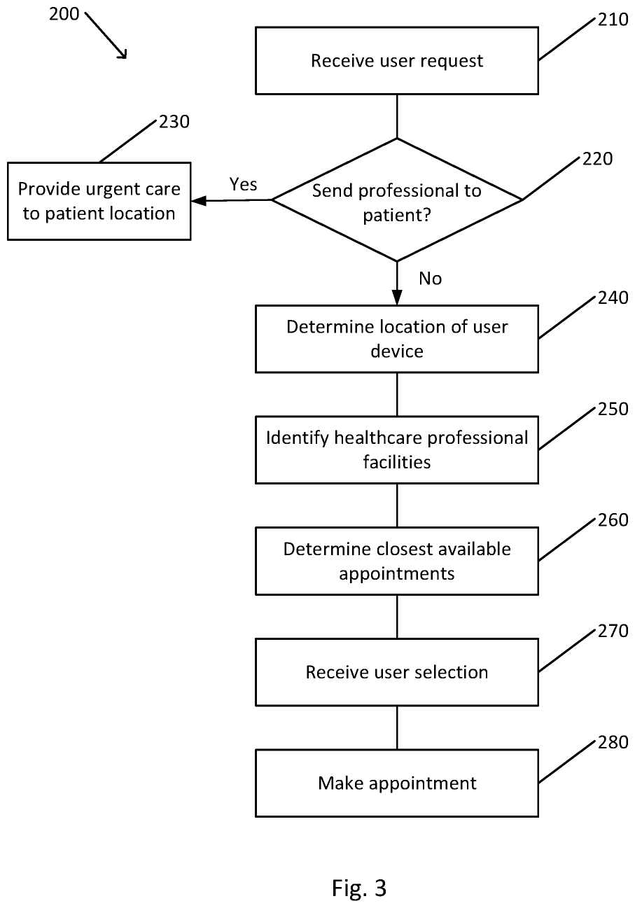

FIG. 3 is a flowchart depicting an example of a method of operation of a server of an urgent care system that includes directing a patient to the location of a healthcare professional.

Urgent care provider method 200 may be executed by a processor of system server 12.

A request for medical assistance from a user mobile device 14 may be received (block 210). For example, the request may include a type of healthcare professional required, or a condition of a patient for which assistance or care is requested.

In some cases, system server 12 may send to user mobile device 14 a request for one or more types of login information. In this case, receipt of the request from user mobile device 14, as well as other operations of urgent care provider method 100, may be blocked or suspended until the login information is received. In some cases, system server 12 may send to user mobile device 14 a request for further information specifying the patient or the patient's condition, which may be received from user mobile device 14. For example, the request for further information may include a request for a more detailed description of the patient's condition, a photograph or video of a part of the patient's body that is related to the patient's condition, a recording of the patient, or other data.

System server 12 may determine, e.g., on the basis of predetermined criteria, whether a healthcare professional is to be sent to the location of the patient or the patient is to be sent to the location of the healthcare professional (block 220). In some cases, system server 12 may send a request to a human supervisor of urgent care provider system 10 to verify the decision or to decide in the case that is not covered by the predetermined criteria. In some cases, a response to user mobile device 14 may include suggested links to websites with additional information related to the patient's condition, or advertisements for various products or services.

When it is decided to send a healthcare professional to the location of the patient, system server 12 may executed the operations of blocks 120 to 170 of urgent healthcare provider method 100 (block 230).

When it is decided to send the patient to the location of the healthcare professional, system server 12 may communicate with user mobile device 14 to determine a location of user mobile device 14 (block 240). In some cases, the user of user mobile device 14 may specify, and system server 12 may receive, a location of a patient that is different from that of user mobile device 14. In this case, further reference to the location of user mobile device 14 in steps of urgent care provider method 200 may refer to the received location of the patient.

System server 12 may identify one or more healthcare professional facilities that are available to provide the requested care and that are registered with the urgent care provider service that operates system server 12 (block 250). For example, system server 12 may select one or more healthcare professionals from healthcare professional database 30 whose profession or specialty corresponds to a profession or specialty that is requested by the user or that is matched with the user (e.g., via a database or algorithm that matches a described condition of the patient to a type of healthcare professional).

A closest available appointment time for each identified healthcare professional facility may be found and sent to user mobile device 14 (block 260). In some cases, one or more additional available appointment times may be found. In some cases, only appointment times that allow sufficient travel time from the location of the patient to the healthcare professional facility may be sent to user mobile device 14. Information regarding each healthcare professional and each healthcare professional facility, may also be provided.

System server 12 may receive from user mobile device 14 an indication of acceptance of one of the proposed appointments (block 270).

System server 12 may then communicate with the healthcare professional facility to reserve the appointment (block 280). System server 12 may send confirmation of the appointment, possibly including travel instructions, to user mobile device 14.

Different embodiments are disclosed herein. Features of certain embodiments may be combined with features of other embodiments; thus, certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An urgent care provider system, the system comprising a server that is configured to:
receive a request for medical assistance for a patient from a user mobile device that is registered with the system;
identify one or a plurality of healthcare professionals that are registered with the system and who can provide the requested medical assistance;
determine a location of the patient;
determine locations of one or a plurality of healthcare professional mobile devices, each healthcare professional mobile device associated with a healthcare professional of the one or a plurality of healthcare professionals;
calculate a travel time between each of the one or a plurality of healthcare professionals and the patient;
present one or a plurality of possibilities of provision of the medical assistance by one or more of the one or a plurality of healthcare professionals; and
if one of the plurality of possibilities is selected, send to the healthcare professional mobile device that is associated with the selected healthcare professional instructions to provide the requested medical assistance to the patient.

2. The system of claim 1, wherein the server is further configured to determine whether to send a healthcare professional of said one or a plurality of healthcare professionals to the location of the patient, or to send the patient to a facility associated with one of the one or a plurality of healthcare professionals.

3. The system of claim 2, wherein the server is configured to determine a time of an available appointment at the facility.

4. The system of claim 1, wherein the server is configured to determine the location of the patient by determining a location of the user mobile device.

5. The system of claim 1, wherein the server is configured to determine the location of the patient by receiving a location that is indicated by operation of the user mobile device.

6. The system of claim 1, wherein the server is configured to present the one or a plurality of possibilities by presenting the request for assistance concurrently to a plurality of the healthcare professional mobile devices.

7. The system of claim 1, wherein the server is configured to present the one or a plurality of possibilities by presenting the request for assistance sequentially to each of a plurality of the healthcare professional mobile devices until the request is via a healthcare professional mobile device of the plurality of the healthcare professional mobile devices.

8. The system of claim 1, wherein the server is configured to present the one or a plurality of possibilities by presenting offers of assistance one or a plurality of the healthcare professionals to the user mobile device.

9. The system of claim 1, wherein the server is further configured to request from the user mobile device data that is related to a medical condition of the patient.

10. The system of claim 9, wherein the data comprises a photograph or a video recording of the patient.

11. The system of claim 9, wherein the data comprises a temperature of the patient.

12. The system of claim 1, wherein the server is further configured to enable payment of a fee for provided medical assistance via the user mobile device.
